(19) United States
(12) Patent Application Publication
(10) Pub. No.: US 2010/0159016 A1
(43) Pub. Date: Jun. 24, 2010

(54) PAIN RELIEF COMPOSITION COMPRISING PARAMAGNETIC SILVER NANOPARTICLES

(75) Inventor: Young-Nam KIM, Wanju-gun (KR)

Correspondence Address: THE WEBB LAW FIRM, P.C., 700 KOPPERS BUILDING, 436 SEVENTH AVENUE, PITTSBURGH, PA, 15219, US

(73) Assignee: Nano Plasma Center Co., Ltd., Wanju-gun (KR)

(21) Appl. No.: 12/718,540

(22) Filed: Mar. 5, 2010

Related U.S. Application Data
(63) Continuation of application No. 11/821,796, filed on Jun. 26, 2007.
Application No. 11/821,796 is a continuation of application No. 10/588,135, filed on Jul. 31, 2006.

(30) Foreign Application Priority Data
Jul. 21, 2006 (KR) 10-2006-0068357

(51) Int. Cl. A61K 9/14; A61K 33/38; A61P 19/02

(52) U.S. Cl. 424/489; 424/618; 977/773; 977/915

(57) ABSTRACT

The present invention relates to a pain relief composition comprising paramagnetic silver nanoparticles. More specifically, the composition is characterized in that it comprises from 0.03 to 0.05% by weight of paramagnetic silver nanoparticles having specific properties that were not revealed by conventional diamagnetic silver nanoparticles. The pain relief composition according to the present invention shows excellent effect of relieving pain of arthritis due to antibiotic property and anti-toxicity of paramagnetic silver nanoparticles. By using the paramagnetic silver nanoparticles having small particle size, the composition is rapidly absorbed into cells upon being applied to skin. In addition, by virtue of using silver (Ag), no complication such as skin coloration or edema was observed. According to the present invention, pain is relieved by simple application, so that the usage is easy as compared to surgical treatment such as arthroscopic operation to provide high satisfaction to a patient.

### CROSS-REFERENCE TO RELATED APPLICATIONS

This application is a continuation application of U.S. patent application Ser. No. 11/821,796, filed Jun. 26, 2007 and a continuation of U.S. patent application Ser. No. 10/588,135, filed Jul. 31, 2006. The entire contents of the above-referenced applications are incorporated herein by reference.

### BACKGROUND OF THE INVENTION

1. Field of the Invention

The present invention relates to a pain relief composition comprising paramagnetic silver nanoparticles.

2. Description of Related Art

Magnetic properties of substances are divided into ferromagnetic, weakly magnetic and diamagnetic substances, and the weakly magnetic substances are classified into semi-ferromagnetic substances and paramagnetic substances. In case of paramagnetic substances, the magnetic effects of electrons in most atoms or ions, including spinning and orbital motions, are exactly offset, so that the atoms or ions do now show magnetic properties. This occurs in an inert gas such as neon or copper ions constituting copper. However, in some atoms or ions, the magnetic effects of electrons are not completely offset, so that the entire atom may have magnetic dipole moment. When n atoms each having magnetic dipole moment are placed in a magnetic field, those atomic dipoles tend to be aligned side by side. Such a tendency is referred to as paramagnetic. If all of them are completely aligned in one direction, the total dipole moment would be nμ. However, the process of alignment is intervened by thermal motion. Due to random vibration of atoms, collisions between the atoms occur, and the kinetic energy is transferred, so that the aligned state is already destroyed. The significance of thermal motion can be seen by comparing the two types of energy. Among them, one is mean translation kinetic energy possessed by an atom at temperature T, (3/2)kT. The other is energy difference between two states (2 μB): parallel state and anti-parallel state to the direction of magnetic field of the magnetic dipoles. At a conventional temperature or magnetic field, the former is significantly larger than the latter. Thus, the thermal motion of atoms has a role to inhibit alignment of the dipoles. Though magnetic moment arises in external magnetic field, it cannot reach the possible maximum nμ by any means. To represent the extent to which any substance is magnetized, considered is magnetic moment per unit volume. This is referred to as magnetized M.

Though a substance called as a diamagnetic does not have inherent magnetic dipole, nor paramagnetism, a magnetic moment can be induced by an external magnetic field. When a sample of such a substance is placed near a strong and uneven magnetic field, magnetic power works. In contrast with an electric case, it is not attracted to the poles of the magnet, but repelled. Such a difference between electricity and magnetism comes from the fact that electric dipole has the same direction as external electric field, while the induced magnetic dipole is in the opposite direction to external magnetic field. Diamagnetism means that Faraday's law of induction is applied to the electrons of an atom, and from the traditional viewpoint, the motion of electrons are very small rings of electric current. The fact that the direction of the induced magnetic moment is opposite to the direction of magnetic field results from Lenz's law at the atomic level.

Diamagnetism is the property possessed by every atom. However, if an atom has inherent magnetic dipole moment, the diamagnetic effect is shielded by the stronger paramagnetism or ferromagnetism.

In the meanwhile, silver is a representative diamagnetic substance, showing the magnetism in an opposite direction to the external magnetic field. The diamagnetic property of silver is known to be unchanged even if the size of silver is made at nano level.

Silver inhibits metabolism of microorganisms to suffocate the microorganisms, by virtue of antibiotic effect of active oxygen. The oxygen bonded to silver in the form of silver oxide is partly converted to active oxygen via catalytic action of silver. Active oxygen has strong antibiotic effect, and shows excellent effect on raising the immunity of a human body. Silver also has the effects of maintaining the balance of hormone system in a human body, blocking electromagnetic wave or water vein wave, releasing far infrared wave or anions, as well as its antibacterial and antifungal activity.

However, conventional diamagnetic silver has poor dispersibility due to high cohesive properties between the particles, so that the application is restricted. Silver nanoparticles have been applied merely in the field based on the natural properties of silver, including cosmetics, textiles, paints, bio-products such as plastics, and antibiotic, antibacterial and soil-resistant substances.

This is because the surface of conventional diamagnetic silver is enclosed by oxidative layers, though the silver powder has reduced volume property with nano-particle size, so that the specific surface properties cannot be revealed, and the diamagnetic property remains unchanged.

Recently, numerous studies have been accomplished by using silver nanoparticles. In particular, they are applied in various fields requiring antibacterial activity due to the antibiotic activity of silver nanoparticles (J. Proteome. Res. 2006; 5: 916-924, J. Am. Chem. Soc. 2005; 127: 2285-2291). It was also reported recently that expression of various cytokines causing inflammation was reduced in patients of skin disorders, when an ointment containing silver nanoparticles was applied, thereby resulting in rapid remedy of skin disorders in experimental animal models (Br. J. Dermatol. 2005; 152: 1235-1242).

It is known that silver nanoparticles readily move into cells by taking advantage of their small size and become located inside the endosomes, as can be seen from FIGS. 1 and 2. Though the discharge route from the human body is not exactly known, the use of topical applications such as toothpastes is already approved by FDA.

In the meanwhile, no pain relief composition comprising paramagnetic silver nanoparticles has been disclosed up to the present.

The present inventors developed silver nanoparticles having paramagnetic property that was not found in conventional silver nanoparticles, and filed as a Korean Patent Application No. 2004-68246. They have further investigated to develop products by using the silver nanoparticles, and found that a pain relief composition comprising silver nanoparticles having paramagnetic property exhibit extremely excellent effect as compared to conventional silver nanoparticles: in that the inventive composition shows stronger anti-toxicity as compared to the composition with conventional diamagnetic silver particles, as well as inherent effect of raising the activity of various active components contained in the pain relief composition; the paramagnetic silver nanoparticles do not cause cohesion in the composition but have excellent dispersibility; and it provides very good effect in terms of antibiotic activity as compared to the composition using conventional silver nanoparticles: and completed the present invention.

### SUMMARY OF THE INVENTION

The object of the invention is to provide a pain relief composition comprising paramagnetic silver nanoparticles, which shows strong anti-toxicity and excellent antibiotic and disinfectant property with rapid absorption to skin, and exhibits enhanced pain relieving effect.

The present invention relates to a pain relief composition comprising paramagnetic silver nanoparticles. More specifically, the composition is characterized in that it comprises from 0.03 to 0.05% by weight of paramagnetic silver nanoparticles having specific properties that were not revealed by conventional diamagnetic silver nanoparticles. The paramagnetic silver nanoparticles exhibit paramagnetism at any temperature range, but particularly at absolute temperature of 20K or higher.

### BRIEF DESCRIPTION OF THE DRAWINGS

FIG. 1 is a photograph of a nanoparticle.

FIG. 2 is a photograph of silver nanoparticles that were absorbed in cells.

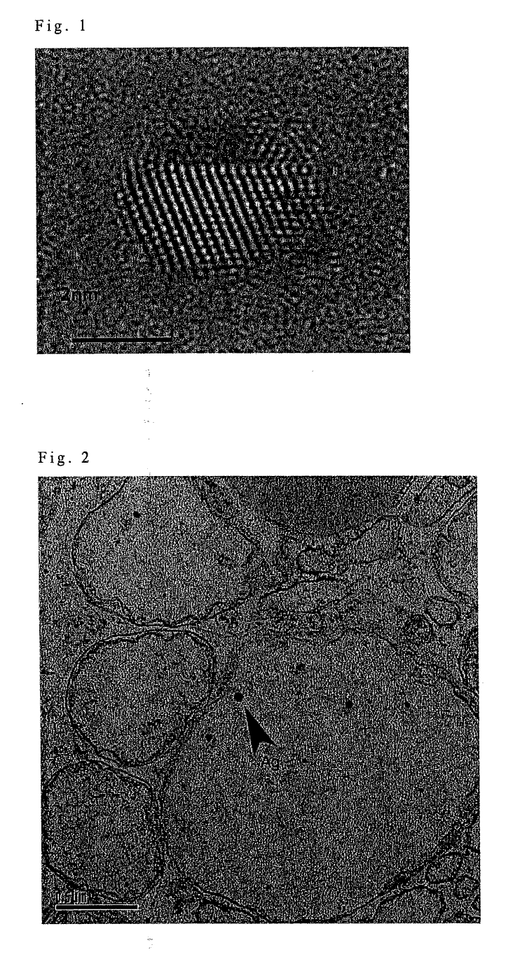

Other and further objects, features and advantages of the invention will appear more fully from the following description.

### BRIEF DESCRIPTION OF THE INVENTION

The pain relief composition according to the present invention is characterized by comprising paramagnetic silver nanoparticles in addition to a conventional pain relief composition. The pain relief composition according to the present invention, due to the paramagnetic silver nanoparticles, shows peculiar effects that were not shown by conventional diamagnetic silver nanoparticles; those are stronger anti-toxicity and enhanced activity of individual active components contained in the pain relief composition. Addition of paramagnetic silver nanoparticles does not cause cohesion, but provides excellent dispersibility, while exhibiting extremely good antibiotic activity as compared to conventional silver nanoparticles.

Paramagnetic silver particles contained in the pain relief composition according to the present invention have paramagnetism at any temperature. This means they have same direction (positive magnetic susceptibility) as external magnetic field at any temperature range. In particular, such paramagnetic feature of silver nanoparticles can be confirmed more clearly at absolute temperature 20K or higher. The paramagnetic silver nanoparticles according to the present invention show distinct properties from conventional nanoparticles in that they show very low coercivity; they don't have surface oxidative layer; they are stable at ambient temperature; they don't have cohesive property; and they have high dispersibility. Conventional diamagnetic silver nanoparticles, though having reduced volume property by virtue of nanotechnology, had problems in that they could not reveal specific properties on the surface because the surface of the nanoparticles are enclosed by oxidative layers.

The paramagnetic silver nanoparticles employed in the pain relief composition according to the present invention reveal different slopes of the magnetic susceptibility curve, depending on the size of the nanoparticles: the smaller the size, the more noticeable paramagnetic property is shown. Silver particles of hollow structure having the empty inside of the particles also show paramagnetic property. Those silver particles show different magnetic susceptibility curves depending on temperature: they exhibit paramagnetic property at a temperature range not more than ambient temperature. The coercivity of silver nanoparticles according to the present invention is 5 gauss or less at a temperature range of not more than ambient temperature. Particularly, the nanoparticles have very small coercivity, as 2 gauss or less, at ambient temperature.

The particle size of the paramagnetic silver nanoparticles employed in the pain relief composition according to the present invention is not specifically restricted, being suitable only if they would be in the size having paramagnetic property. Since the paramagnetic property of silver nanoparticles employed in the pain relief composition of the present invention occurs when the particle size is in a range of not more than 40 μm, the size of not more than 40 μm is preferable. As the slope of magnetic susceptibility curve depends on the size of silver nanoparticles, paramagnetic property is eminently revealed with the particles having the size within the range mentioned above, and the particles having the size of 20 to 50 nm are more preferable. The smaller the size of the silver nanoparticles, the more distinct the paramagnetic property is, so that higher effects of the present invention are shown. However, the production cost increases for smaller particles, and the inventive effects are sufficiently shown with the size in the above range, so that it is not necessary to use even smaller particles than those mentioned above.

The amount of paramagnetic silver nanoparticles contained in the pain relief composition of the present invention partly varies depending upon the paramagnetic property of silver. The content of paramagnetic silver nanoparticles preferably is from 0.03 to 0.05% by weight, based on the size of not less than 40 μm, preferably from 20 to 50 nm, and coercivity of not more than 5 gauss.

As a result of toxicity test of the paramagnetic silver nanoparticles employed in the present invention, wherein an aqueous dispersion of 0.03% of the paramagnetic silver nanoparticles is orally administered to SD rats at a critical amount of 20 ml/kg body weight, toxicity symptom or histopathologic lesion was not found at all. Thus the amount of the paramagnetic silver nanoparticles in the pain relief composition according to the present invention is not restricted, because they have no harm to human body. If the amount of the silver nanoparticles is less than 0.03% by weight, desired effect may be insufficient. If the amount is more than 0.05% by weight, it is disadvantageous in view of economy since the effect of adding the paramagnetic silver nanoparticles does not increase any more.

Specific features of the paramagnetic silver nanoparticles employed in the pain relief composition according to the present invention and a process for preparing the same are disclosed in detail by Korean Patent Application No. 2004-68246 entitled as “Gold or Silver Powder Having Paramagnetic Property”, which was filed by the present inventors. Preparation of the nanoparticles or affirming the features can be readily understood and practiced by a person having ordinary skill in the field of producing nanoparticles on the basis of the patent application mentioned above, so that the relevant explanation is omitted herein.

The pain relief composition according to the present invention comprises the paramagnetic silver nanoparticles, and one or more component(s) selected from mica, silicon dioxide, titanium dioxide and germanium dioxide, with vaseline (petrolatum) as a conventional ointment base.

Vaseline is a semi-solid hydrocarbon wax, such as yellow vaseline, white vaseline and a mixture thereof. Vaseline is used in an amount of 60 to 65% by weight on the basis of the pain relief composition.

In the pain relief composition according to the present invention, one or more component(s) selected from mica, silicon dioxide, titanium dioxide and germanium dioxide, contained along with the paramagnetic silver nanoparticles is (are) employed within the range of 10 to 30% by weight on the basis of total pain relief composition.

Mica serves to retain skin moisture. Mica particles of the size of from 10 to 30 μm are employed in an amount of 5 to 7% by weight on the basis of total pain relief composition.

Silicon dioxide serves as an anti-cohesive agent to avoid aggregation of the components, an excipient and a lubricant. Silicon dioxide having the particle size of 1 to 3 μm is employed in an amount of 3 to 5% by weight on the basis of total pain relief composition.

Titanium dioxide serves as a brighter that provides white color. Titanium dioxide having the particle size of 50 nm to 3 μm is employed in an amount of 1 to 3% by weight on the basis of total pain relief composition.

Germanium dioxide gives immunological enhancement in a human body, which proliferates T-lymphocytes to protect human body from cancer cells, toxic substances, virus, or the like. It is also reported that germanium dioxide causes generation of interferon, one of the BRM (Biological Response Modifier) produced by cells, in human body. However, due to its low solubility, germanium dioxide is troublesome in providing sufficient immunological enhancement in human body. However, when it is contained in the composition along with the paramagnetic silver nanoparticles according to the present invention, the effect of immunological enhancement is increased in human body.

Germanium dioxide, just like the paramagnetic silver nanoparticles, gives no harm to human body, so that the amount to be used is not restricted. The content of germanium dioxide is preferably from 0.5 to 7% by weight on the basis of total pain relief composition.

Germanium doxide contained in the pain relief composition according to the present invention is an extract of natural lignite (an organic substance), which is powder of high purity obtained from combustion of lignite at a high temperature between 1600° C. and 2000° C., and washing with water.

The germanium dioxide obtained from natural lignite extract, which is employed in the present invention, has different crystal structure from conventional germanium dioxide obtained from oxidation of germanium chloride, thereby showing excellent dispersibility in water and stability after dispersion, as well as good reactivity with paramagnetic silver nanoparticles.

As a result of toxicity test of germanium dioxide obtained from lignite according to the present invention, wherein an aqueous dispersion of 3% of the germanium dioxide is orally administered to SD rats at a critical amount of 20 ml/kg body weight, toxicity symptom or histopathologic lesion was not found at all.

Additional ingredients that can be contained in the composition include one or more components selected from a diluent, a moisturizer, a surfactant and an aroma.

The diluent serves to dilute the pain relief composition into a suitable concentration and to intimately dissolve the composition. A hydrophilic diluent is preferably used in the composition according to the present invention. Preferable hydrophilic diluents include water, C1-C4 monohydric alcohol, and glycols and polyols having low molecular weight, such as propylene glycol, polyethylene glycol, polypropylene glycol, glycerol, butylene glycol, 1,2,4-butantriol, sorbitol ester, 1,2,6-hexantriol, ethanol, iso-propanol, ethoxylated ether and propoxylated ether. Any conventionally used diluents may be employed in the composition according to the present invention. The amount of the diluent to be used is from 2 to 10% by weight.

The moisturizer serves to enhance moisture-retaining ability of skin. As the moisturizer, employed is (are) one or more compound(s) selected from ethylene glycol, propylene glycol, dipropylene glycol, butylene glycol, polypropylene glycol, polyethylene glycol, sorbitol, hydroxypropyl sorbitol, ethythritol, threitol, pentaerythritol, xylitol, glucitol, mannitol, hexylene glycol, glycerine, conc. glycerine, sodium hyaluronate, cyclomethicone and dimethicone, or a mixture thereof. The amount of the moisturizer used is from 5 to 30% by weight. If an amount less than that is employed, the moisturizing effect is insignificant. Even if the amount more than that is used, further increase of moisture-retaining ability rarely occurs.

The surfactant serves to disperse and suspense discrete phases in a continuous phase. Any conventional surfactants may be used, as long as the selected surfactant is chemically and physically compatible with other constituents of the composition and satisfies required properties. Preferable surfactants include polyacrylamide, ester oil, isohexadecane, cetanol, stearyl alcohol, oleyl alcohol, lauric acid, myristic acid, palmitic acid, dimethyloctanoic acid, or the like. The amount of the surfactant preferably is from 8 to 10% by weight. If the amount is less than that, soft feeling may be reduced owing to lack of oily components. If the amount is more than that, the problem of excessively oily feeling may occur.

As the aroma, employed can be one substance selected from menthol, eucalyptol, anetol, peppermint oil, spearmint oil, sage, methyl salicylate and fruit extracts, or a mixture thereof.

The pain relief composition according to the present invention is characterized in that it relieves pain of arthritis with the effect of regeneration of cartilage in degenerative arthritis, when being applied to the site of arthritis.

The pain relief composition according to the present invention is prepared according to a process for preparing conventional ointment preparation, by the use of said pain relief composition. In other word, the composition is prepared by melting the components other than the paramagnetic silver nanoparticles, and then mixing the paramagnetic silver nanoparticles into the molten components under stirring.

### EXAMPLES

Now the present invention is explained more specifically by means Examples, which are provided for illustration only, but are not intended to limit the scope of the present invention by any means.

The paramagnetic silver nanoparticles added are those prepared according to Preparation Example 3 of Korean Patent Application No. 2004-68246.

### Example 1

Pain relief compositions were prepared according to conventional process for preparing an ointment by using the component ratio as shown in Table 1:

TABLE 1

| Components (wt %) | | Example 1 |
|---|---|---|
| Paramagnetic silver nanoparticles | | 0.05 |
| Mica (15 μm) | | 5.5 |
| Silicon dioxide (2 μm) | | 4.5 |
| Titanium dioxide (1 μm) | | 2 |
| Germanium dioxide (1 μm) | | 2.45 |
| Base | Vaseline | 60 |
| Surfactant | Polyacrylamide | 8 |
| | (Sepigel 305, from Acofarma) | |
| Diluent | Butylene glycol | 3.5 |
| Moisturizer | Sorbitol | 5 |
| | Cyclomethicone | 2 |
| | Xylitol | 2 |
| Aroma | L-menthol | 5 |

### Experimental Example 1

### Examination of Pain Relieving Activity

Effect of improving the pain relieving activity in temporomandibular joint (TMJ) capsulitis was examined by once applying the pain relief composition comprising paramagnetic silver nanoparticles according to Example 1 of the present invention to skin where the joint lateral capsular ligament of preauricular region exists, of 49 patients who had been diagnosed as TMJ capsulitis.

The patients who had been diagnosed as TMJ capsulitis are listed in Table 2. The patients consisted of 20 male patients (40.8%) and 29 femail patients (59.2%). Among them, the number of patients of acute TMJ capsulitis having the retention period of the symptoms less than 4 weeks was 26 (53.1%), while the number of patients of chronic TMJ capsulitis having the period of more than 4 weeks was 23(46.9%). There were 16 patients accompanied with retrodiscitis, 2 patients with internal derangement of temporomandibular joint (TMJ ID), and one patient with osteoarthritis.

The patients are divided into two groups: Group A (30 persons) and Group B (19 persons). To the patients of Group A, the pain relief composition comprising paramagnetic silver nanoparticles from Example 1 was once applied to the skin where the joint lateral capsular ligament of preauricular region exists. To examine the placebo effect, the ointment of Example 1 but excluding the paramagnetic silver nanoparticles was once applied to the patients of Group B.

The pain relief effect on TMJ capsulitis was measured by referring to the article published before (J. Oral Maxillofac. Surg. 2002; 60: 797-803, Oral Surg. Oral Med. Oral Pathol. Oral Radiol. Endodon. 2005; 99: 677-681), from the aspect of maximum mouth opening, visual analogue scale (VAS) for pain and VAS for function, at the time of first medical examination and 24 hours after the application of the pain relief composition. In order to avoid the evaluation of VAS for pain and function with identical value of the patient's own accord; the VAS for pain was evaluated based on a scale where 10 means most severe pain, while 0 means no pain; and the VAS for function where 10 means no discomfort to the joint function, while 0 means very severe discomfort at the joint. In statistic handling of individual data, the difference of improvement in individual parameter was compared by means of independent sample t-test. The statistically significant level is defined as p<0.05.

The values of individual parameters measured in Group A (Experimental group) and B (Control group) are shown in Table 3.

TABLE 2

| | | Number of patients (%) |
|---|---|---|
| Sex | Female | 29 (59.2%) |
| | Male | 20 (40.8%) |
| Affected joint | Right | 9 (18.4%) |
| | Left | 30 (61.2%) |
| | Both | 10 (20.4%) |
| Retention period | Less than 1 week | 16 (32.7%) |
| of symptoms | From 1 week to 4 weeks | 10 (20.4%) |
| | More than 4 weeks | 23 (46.9%) |
| Retrodiscitis | With | 16 (32.7%) |
| | Without | 33 (67.3%) |
| TMJ ID | With | 2 (4.1%) |
| | Without | 47 (95.9%) |
| Osteoarthritis | With | 1 (2.0%) |
| | Without | 48 (98.0%) |

TABLE 3

| | | Exp. group | Control group |
|---|---|---|---|
| | | Group A | Group B |
| | | (30 persons) | (19 persons) |
| Average age | | 32.50 ± 10.61 | 34.42 ± 10.44 |
| TMJ capsulitis | Acute | 17 | 9 |
| | Chronic | 13 | 10 |
| Maximum mouth | Pre | 37.13 ± 7.55 | 37.21 ± 4.14 |
| opening (mm) | Pol | 41.00 ± 7.18 | 38.42 ± 4.15 |
| VAS for pain (point) | Pre | 4.83 ± 1.96 | 3.24 ± 1.49 |
| | Pol | 2.66 ± 2.28 | 3.09 ± 1.63 |
| VAS for function (point) | Pre | 5.48 ± 2.68 | 6.03 ± 1.59 |
| | Pol | 6.62 ± 2.66 | 8.22 ± 9.05 |

As can be seen from Table 3, average age of patients belonging to Group A (Experimental group) was 32.50±10.61, with no statistically significant difference from the average age of 34.42±10.44 of Group B (Control group) (p>0.05). Pretreatment maximum mouth opening was 37.13±7.55 mm for Group A, and 37.21±4.14 mm for Group B. The post-treatment maximum mouth opening 24 hours after the application of the pain relief composition was 41.00±7.18 mm for Group A, and 38.42±4.15 mm for Group B. Both pretreatment and post-treatment maximum mouth opening values of Group A and B were compared, but no significant difference was recognized (p>0.05).

The pretreatment joint functions of Group A and B were 5.48±2.68 and 6.03±1.59, respectively. Twenty four hours after the application of the pain relief composition, Group A and B revealed functionally improved post-treatment values (6.62±2.66 and 8.22±9.05, respectively) as compared to the pretreatment values, but there was no significant difference (p>0.05) between them.

The pretreatment VAS values for joint pain of Group A and B were 4.83±1.96 and 3.24±1.49, respectively, while the same values 24 hours after the application of the pain relief composition were 2.66±2.28 and 3.09±1.63, respectively. No statistically significant difference was recognized between the pretreatment and post-treatment values (p>0.05).

However, in the Group A (Experimental group), the comparison of pretreatment value with the post-treatment value exhibited statistically significant difference (p<0.05). Group A was divided into the acute TMJ capsulitis group and the chronic TMJ capsulitis group, and the improvements were compared. The results are shown in Table 4 and 5.

TABLE 4
Comparison of improvements in acute TMJ capsulitis

| | Group A | Group B | |
|---|---|---|---|
| | (17 patients) | (9 patients) | p-values |
| Improvement in maximum | 4.06 ± 4.52 | 0.67 ± 1.66 | 0.041 |
| mouth opening (mm) | | | |
| Improvement in VAS of | 2.62 ± 1.85 | 0.08 ± 0.31 | <0.001 |
| joint pain | | | |
| Improvement in VAS of | 1.17 ± 1.44 | 4.46 ± 13.44 | NS |
| joint function | | | |

As can be seen from Table 4, for the patients diagnosed as acute TMJ capsulitis belonging to A group, the average improvement of maximum mouth opening 24 hours after the application of the pain relief composition was 4.06±4.52 mm, while for the patients in Group B, the improvement was 0.67±1.66 mm. The difference of improvement between two groups was recognized significant (p=0.041). The improvement values of pain were 2.62±1.85 for Group A, and 0.08±0.31 for Group B. Prominent improvement in pain relief was recognized in Group A as compared to Group B (p<0.001). However, from the aspect of function, Group B (4.46±13.44) showed improvement as compared to Group A (1.17±1.44), but the result was not statistically significant (p>0.05).

TABLE 5
Comparison of improvements in chronic TMJ capsulitis

| | Group A | Group B | |
|---|---|---|---|
| | (13 patients) | (10 patients) | p-values |
| Improvement in maximum | 3.62 ± 3.33 | 1.70 ± 3.37 | NS |
| mouth opening (mm) | | | |
| Improvement in VAS of | 1.57 ± 2.04 | 0.22 ± 0.74 | NS |
| joint pain | | | |
| Improvement in VAS of | 1.10 ± 1.69 | 0.16 ± 0.40 | 0.042 |
| joint function | | | |

As can be seen from Table 5, for the patients diagnosed as chronic TMJ capsulitis belonging to A group, the average improvement of maximum mouth opening 24 hours after the application of the pain relief composition was 3.62±3.33 mm, while for the patients in Group B, the improvement was 1.70±3.37 mm. Though the average mouth opening value of Group A was higher than that of Group B (Control group), statistical comparison of the improvement of the two groups showed no statistical significance (p>0.05). The improvement values of pain were 1.57±2.04 for Group A, and 0.22±0.74 for Group B. No statically significant difference was found (p>0.05). However, from the aspect of function, Group A (1.10±1.69) showed noticeable improvement of function as compared to Group B (0.16±0.40), with statistical significance (p=0.042).

From the results shown in Tables 3, 4 and 5, it is found that the pain relief composition comprising paramagnetic silver nanoparticles according to the present invention relieves pain by once applying the composition on skin having TMJ capsulitis, due to the anti-toxic effect, antibiotic ability, and rapid cell-absorption (resulting from small particle size) of silver (Ag) nanoparticles. Only single application of the composition provided sufficient pain relieving effect for acute TMJ capsulitis, though the pain relief composition according to the invention did not provide pain relief effect for chronic TMJ capsulitis.

The pain relief composition comprising paramagnetic silver nanoparticles according to the present invention shows excellent effect to relieve pain of arthritis due to the antibiotic property and anti-toxicity of paramagnetic silver nanoparticles, as well as effect of cartilage regeneration in degenerative arthritis. In addition, by using the paramagnetic silver nanoparticles having small particle size, the composition is rapidly absorbed into cells upon being applied to skin. Further, by virtue of using silver (Ag), no complication such as skin coloration or edema was observed. According to the present invention, pain can be relieved by simple application, so that the usage is easy as compared to surgical treatment such as arthroscopic operation, to provide high satisfaction to a patient.

The invention claimed is:

1. A pain relief composition comprising paramagnetic silver nanoparticles and one or more component(s) selected from mica, silicon dioxide, titanium dioxide and germanium dioxide.

2. The pain relief composition according to claim 1, wherein said paramagnetic silver nanoparticles are contained in an amount of 0.03 to 0.05% by weight based on the total pain relief composition.

3. The pain relief composition according to claim 1, wherein said paramagnetic silver nanoparticles have paramagnetism at absolute temperature of 20K or higher.

4. The pain relief composition according to claim 1, wherein the size of said paramagnetic silver nanoparticles is from 20 to 50 nm.

5. The pain relief composition according to claim 1, wherein the total amount of said paramagnetic silver nanoparticles and said one or more component(s) selected from mica, silicon dioxide, titanium dioxide and germanium dioxide is from 10 to 30% by weight based on the total pain relief composition.

6. The pain relief composition according to claim 1, which comprises from 5 to 7% by weight of mica, from 3 to 5% by weight of silicon dioxide, from 1 to 3% by weight of titanium dioxide and from 0.5 to 7% by weight of germanium dioxide.

7. The pain relief composition according to claim 1, which additionally comprises one or more substance(s) selected from diluents, moisturizers, surfactants and aromas.

8. The pain relief composition according to claim 1, which relieves the pain of arthritis.

9. The pain relief composition according to claim 2, which relieves the pain of arthritis.

10. The pain relief composition according to claim 3, which relieves the pain of arthritis.

11. The pain relief composition according to claim 4, which relieves the pain of arthritis.

12. The pain relief composition according to claim 5, which relieves the pain of arthritis.

13. The pain relief composition according to claim 6, which relieves the pain of arthritis.

14. The pain relief composition according to claim 7, which relieves the pain of arthritis.
